(19) United States
(12) Patent Application Publication
(10) Pub. No.: US 2004/0191899 A1
(43) Pub. Date: Sep. 30, 2004

(54) Method for disrupting cells that lack a cell wall

(76) Inventors: Charbel Haber, Erdenheim, PA (US); Russel J. Lander, Lansdale, PA (US); Jennifer Chen, Harleysville, PA (US)

Correspondence Address: MERCK AND CO INC, P O BOX 2000, RAHWAY, NJ, 070650907

(21) Appl. No.: 10/808,682

(22) Filed: Mar. 25, 2004

Related U.S. Application Data
(60) Provisional application No. 60/457,975, filed on Mar. 27, 2003.

(51) Int. Cl. C12N 5/06; C12N 5/08; C12N 5/00

(52) U.S. Cl. 435/358; 435/366; 435/404

(57) ABSTRACT

The present invention relates to a method for disrupting cultured cells that lack a cell wall, the method comprising passing the cells suspended in a suspension fluid through a nozzle at a low pressure, wherein the outflow of the nozzle does not impinge on the outflow of a second nozzle if multiple nozzles are present.

### CROSS-REFERENCE TO RELATED APPLICATIONS

The present application claims benefit of U.S. provisional application 60/457,975 filed Mar. 27, 2003. The provisional application is incorporated by reference.

### BACKGROUND OF THE INVENTION

Biotechnological and cell culture processes often require large amounts of mammalian cells to be grown in bioreactors, and then disrupted to release the desirable products. Popular techniques used to disrupt mammalian cells include solubilization of the membrane via detergent lysis (SDS, TritonX-100), osmotic pressure which covers chemical lysis (MgCl₂, chaotropes), and mechanical or shear-induced lysis (sonication, homogenization, microfluidization, bead milling, freeze-thaw, and jet impingement). Many protocols favor mechanical disruption processes since it is highly desirable to eliminate the need for the addition and consequent difficult removal of reagents (detergents, enzymes, or osmolarity effectors) and avoid difficulties to scale up physical methods such as freeze/thaw.

A number of mechanical methods have been developed to disrupt cells of microorganisms, which are also common host cells of choice for biotechnological and fermentation processes. These methods generally rely on fluid shear and/or mechanical impact to rupture the cell wall and membrane. However, many of the commercial systems designed for microbial systems are not suitable for use with animal cells. When used to disrupt mammalian cells, those devices may also bring about damage to the desired end-products. Mammalian cells are more susceptible to lysis than bacterial and fungal cells because they are larger and lack the protective cell wall present in bacteria and fungi. Hence, much less energy input is required for the desired cell disruption.

Devices for disrupting bacterial and fungal cells could be modified to become suitable for disrupting mammalian cells. One of them is the impinging jet, which disrupts cells by the impingement of two opposing fluid jets. The impinging region produces a micro-mixing zone where the shear is controlled by the linear velocity of the jet. These devices were first used for the disruption of microbial cells. For example, MICROFLUIDIZER®, a commercial impinging jet device, uses an interaction chamber where two jet streams impinge on one another at linear velocities up to 200+ m/s, at high pressures (about 15,000 to 40,000 psi). The mode of disruption was reported as cavitation, fluid shear and impact. However, this device is not suitable for use with animal cells because not only are cells disrupted, but their contents are damaged as well, even at 2,000 psi.

U.S. Pat. No. 5,721,120 disclosed that the impinging jet process can be used for disrupting animal cells, when the pressure is decreased to less than about 150 psi. When used at the low pressures, the impinging jet process provides adequate disruption of animal cells with negligible loss of cell contents. Although the impinging jet can be used to disrupt mammalian cells at the low pressures, its configuration is still complex. The jet impinger structure engenders many problems in manufacturing, such as the difficulties to scale-up, control, and clean the device.

It would be desirable to have a simplified and non-invasive version which efficiently disrupts animal cells and releases intact and biologically active cell contents.

### SUMMARY OF THE INVENTION

The present invention relates to a method for disrupting cultured cells that lack a cell wall, the method comprising passing the cells suspended in a suspension fluid through a nozzle at a low pressure, wherein the outflow of the nozzle does not impinge on the outflow of a second nozzle if multiple nozzles are present.

According to an embodiment of the present invention, the low pressure used in the cell-disruption method ranges from 1 to 100 psi, preferably from 5 to 70 psi, or further preferably from 10 to 60 psi.

According to an embodiment of the present invention, the nozzle used in the cell-disruption method has an orifice with a diameter ranging from 0.1 mm to 100 mm, preferably from 0.5 mm to 10 mm, or further preferably from 1 mm to 3 mm.

According to an embodiment of the present invention, the low pressure used in the cell-disruption method ranges from 1 to 100 psi, preferably from 5 to 70 psi, or further preferably from 10 to 60 psi.

According to an embodiment of the present invention, the nozzle used in the cell-disruption method has a tapered or conical shape. The nozzle preferably has both a tapered entrance and a tapered exit.

According to an embodiment of the present invention, the cells disrupted in the method are animal cells. The animal cells can be selected, for example, from the group consisting of: VERO cells, CHO cells, and diploid fibroblast cells. The animal cells are preferably MRC-5 diploid lung cells.

The present invention also relates to a method of harvesting a cell product contained within cells that do not have a cell wall. The method comprises, culturing the cells in a culture medium under culture conditions suited to bring about the production of the desired cell product; suspending the cells in a suspension fluid; passing the suspended cells through a nozzle at a low pressure, wherein the outflow of the nozzle does not impinge on the outflow of a second nozzle or any other impingement surface, so that the cells are disrupted at a pressure of from about 5 to 100 psi and the cell product is released; and recovering the released cell product.

According to an embodiment of the present invention, the product harvested in the method selected from a group consisting of a polysaccharide, a protein, and a virus.

The present invention further relates to a method of harvesting a virus grown in an animal cell. The method comprises, culturing animal cells infected with the virus; suspending the animal cells containing the virus in a suspension fluid; passing the suspended animal cells through a nozzle at a low pressure, wherein the outflow of the nozzle does not impinge on the outflow of a second nozzle if multiple nozzles are present, so that cells are disrupted and the virus is released; and harvesting the released virus.

According to an embodiment of the present invention, the virus harvested in the method is Varicella virus.

### BRIEF DESCRIPTION OF THE DRAWINGS

FIG. 1. Schematic of a Single Nozzle Assembly. A nozzle is connected via stainless-steel tubing to a pressurized bio-reactor. The outlet of the nozzle is directed either to a receiving tank or to another unit operation such as a filtration unit or centrifuge.

FIG. 2. An illustration of a Single Nozzle.

FIG. 3. Comparison of Adenovirus (AdV) released from cells lysed by impinging jet and single nozzle device. AdV released is determined by an anion-exchange-chromatography assay. The line on the top indicates the maximum release, which achieved by 0.1% TritonX-100.

FIG. 4. Cells lysed by a single nozzle device with three different orifice diameters: 1, 2.1, and 3 mm. AdV released is determined by an anion exchange chromatography-based assay. The line on the top indicates the maximum release, which achieved by 0.1% TritonX-100.

### DETAILED DESCRIPTION OF THE INVENTION

The present invention relates to a novel, non-invasive device for disrupting cells that do not have a cell wall. A device according to the present invention can be used to disrupt animal cells at low pressure.

The present invention provides a device for cell disruption, which requires only a single nozzle.

The present invention provides a method using a device that disrupts cells at low pressures without the need for impingement of two separate fluid streams.

I. The Structure of the Novel Device for Disrupting Cells

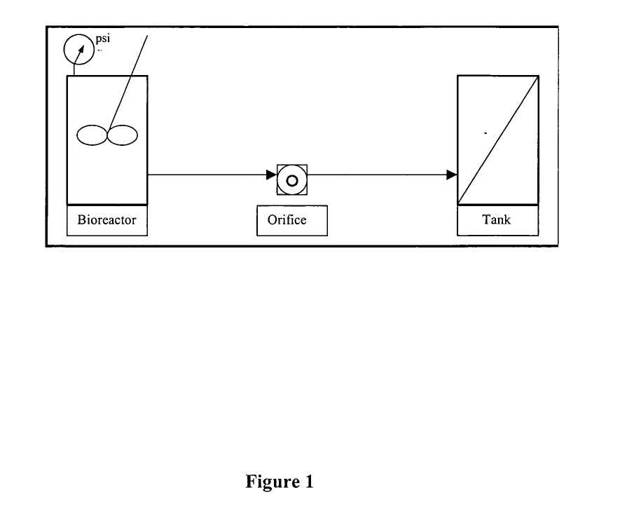

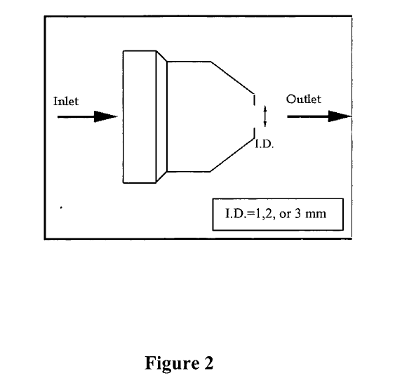

The present invention provides a cell disruption device comprising a nozzle. FIG. 1 and FIG. 2, provides examples of a cell-disruption device comprising a single nozzle for fluid flow. According to a preferred embodiment of the present invention, the cell-disruption device comprises a single nozzle. Thus, the device can be called “single nozzle device” to distinguish from the impinging jet device, which has two nozzles in close proximity and in an opposed flow configuration. Impingement is produced by the fluid flowing from the two opposing nozzles. As used herein, “impingement” refers to the collision of two countercurrent jet streams.

With only one nozzle, a device according to the present invention is not capable of producing any impingement of opposing fluid flows, as the impinging jet device does. Thus, a device according to the present invention is distinct in design from the impinging jet device.

According to an alternative embodiment of the present application, the cell disruption device comprises more than one nozzle, as long as the nozzles are so positioned that the fluid flows out of the nozzles do not impinge at low pressures. To avoid the impingement, the nozzles are preferably oriented in the same direction.

As used herein, “low pressures” refers to the pressures that that make the device run in a non-cavitating mode, preferably less than about 150 psi, and more preferably less than about 100 psi.

According to an embodiment of the present invention, the nozzle has an orifice with a diameter at the range of from 0.1 to 100 mm. According to a preferred embodiment of the present invention, the orifice has a diameter range from 0.5 to 10 mm. According to a further preferred embodiment of the present invention, the orifice has a diameter range from 1 to 3 mm. Most preferably, the orifice has a diameter of 3 mm.

The nozzle used in a cell-disruption device according to the present invention can have different shapes. The nozzle preferably has one of the shapes of nozzles in impinging jet devices. According to a preferred embodiment of the present invention, the nozzle has a tapered or conical shape, as shown in FIG. 2. According to a further preferred embodiment of the present invention, the nozzle has both a tapered entrance and a tapered exit, e.g., a Venturi shape. It is also possible to design the downstream section in a reverse conical fashion to prevent flow separation.

According to an embodiment of the present invention, the cell-disruption device further comprises a pressure regulator, a pressure tank, and a receiving tank.

As shown in FIG. 1, the nozzle was connected to the end of a pressure tank equipped with a pressure regulator. Nitrogen was used to pressurize the tank to a specific pressure. The corresponding flow of the liquid contained in the tank is channeled through the orifice of the nozzle. The nozzle acts as a reducer and transforms the flow into a streaming jet. The effluent is directed into a receiving tank or to another unit operation.

II. The Disruption of Mammalian Cells Using the Novel Device

The present invention provides a novel method of disrupting cultured cells which lack a cell wall comprising passing cells suspended in a suspension fluid through a nozzle at a low pressure.

As shown in FIG. 1, cells that lack a cell wall can be suspended in a suspension fluid, and stored in the pressure tank of the bioreactor. At a low pressure controlled by the pressure regulator, the suspension fluid containing the cells flows out the tank via a tubing connection. The nozzle, through which the pressure is reduced and the cells are disrupted, is connected at the end of the tube. Fluid exiting from the nozzle is transferred to the receiving tank directly, or through a second length of tubing as shown in FIG. 1.

As described above, the device is operated at a low pressure, in a non-cavitating mode, preferably less than about 150 psi, and more preferably less than about 100 psi. The low operating pressure results in a gentle disruption: cells are preferably ruptured at a very low pressure, from about 5 to about 100 psi. If the receiving tank is open to the air, the pressure drop should be equal to the low pressure effected in the pressure tank. According to an embodiment of the present invention, the pressure drop ranges from 1 to 100 psi. According to a further preferred embodiment of the present invention, the pressure drop ranges from 5 to 70 psi. Most preferably, the pressure drop is 60 psi. The pressure drop can be simply controlled by controlling the pressure effected in the pressure tank with the pressure regulator.

In this process, there is no impingement of the flows of the cell suspension fluid, as what is created in the impinging jet device. It appears that the impingement was thought to be essential for the disruption of cells that lack a cell wall at low pressures, because it was preserved in the cell-disruption device of U.S. Pat. No. 5,721,120. In contrast, as shown in example 1, the pressure drop experienced by the suspension fluid flowing through the nozzle is sufficient to disrupt the cells. In other words, the impingement is unnecessary for disrupting the cells and might be even harmful for the contents released from the disrupted cells.

Without the need to create the impingement of flows, a cell disruption device according to the present invention is significantly simplified in comparison with the impinging jet device. In the impinging jet device, the suspension fluid needs to be first divided into two flows, and the two flows via two nozzles at opposing positions impinge. As shown in FIG. 1 and FIG. 2, in a device according to the present invention, the flow of the suspension fluid does not need to be divided, and only one nozzle is needed, because no impingement of flows is created.

Indeed, the advantages of a cell-disruption method according to the present invention are attributed to the simplicity of the cell-disruption device used in the method. One of the advantages of the device over the jet impingement device is the ease of implementation. A device according to the present invention cuts down the number of process controls required to ensure equal distribution of flow in each individual jet, especially if multiple impingers are needed to lyse large process volumes. In addition, the single nozzle is inherently easier to clean, since there are no backflow regions of stagnation points as the impinging jet device has. The device is sanitary in design and can use commercially available nozzle technology for consistent fabrication, and can be incorporated directly into standard process equipment. Further, the device can be sterilized-in-place.

In addition, a device according to the present invention can be quite small in size so it can be incorporated into standard process piping connecting two vessels.

III. The Applications of the Device and the Method

Using a device according to the present invention, cells that lack a cell wall can be disrupted while the contents of the cells are preserved. Thus, another aspect of this invention is a method of harvesting a cell product contained within a cell that does not have a cell wall comprising:

culturing cells under culture conditions in a culture medium until the cell product is produced;

suspending the cells in a suspension fluid;

passing the suspended cells through a nozzle so that the cells are disrupted at a pressure of from about 5 to 100 psi and the cell product is released; and recovering the released cell product.

The device is preferably operated in a continuous single pass mode. The method of this invention may be broadly applied to any cell that lacks a cell wall or that has had its cell wall removed. While mammalian cells are preferred, this method works equally well with other cells, such as plant or fungal protoplasts and bacterial spheroplasts. The only requirement is that the cells be amenable to cell culture, and it is preferred that the cells be amenable to a large scale culture.

Examples of suitable animal cells include VERO cells, CHO cells, and diploid fibroblast cells such as MRC-5 cells. Examples of suitable plant protoplasts include Nicotiana, Petunia, Zea, Brassica, and interspecial hybrids.

The cells lines may be immortalized or not, and they may be cultivated in either a stationary or in a suspension culture. None of the particular culture parameters are critical to the method of this invention.

The method of this invention may be used to recover virtually any type of product that is made using the cultured cells. Examples of such products include: polysaccharides; proteins of different sizes and functions such as antibodies and enzymes; and viruses. Proteins that can be recovered include those naturally produced by a cell and those produced by a modified cell (e.g., a cell containing recombinant nucleic acid encoding a protein), and those produced by a cell infected with a virus or other type of vector. In a preferred embodiment of this invention, animal cells are used as host cells for the production of viruses that are used in the manufacture of vaccines.

In one particularly preferred embodiment, MRC-5 human diploid lung cells infected with Varicella Zoster Virus, particularly with the Oka strain of Varicella Zoster Virus, are disrupted to harvest virus used to prepare a live virus vaccine, VARIVAX®.

The cells can be cultured as is customary for the particular cell. After a suitable culture period, the cells are released from their substrate (if they are anchored), suspended in a fluid. The fluid may be the same or similar to that used to culture the cells, or it may be a stabilizer. For purposes of this invention, the composition of the suspension fluid is not critical. Next, the suspended cells are processed through the nozzle orifice of a cell-disruption device according to the present invention as described above.

A method according to the present invention has been shown to provide adequate cell breakage for high filtration yields with negligible loss of infectious titer.

In a preferred embodiment, the cell disruption method of this invention can be used for high yield recovery of Varicella Zoster Virus, other viruses or intracellular proteins from animal cells. After disruption, cell debris is separated from the associated virus particles by clarifying filter, and the resultant virus preparation is frozen until further processing into the vaccine.

The following examples are according to the way of illustration and are not intended to limit the scope of the present invention.

### EXAMPLES

### Example 1

### Comparison of Impinging Jet and Single Nozzle Device in their Capabilities to Disrupt Mammalian Cells

The geometry of the device used to generate substantial shear to rupture cells is important from a practical perspective. The geometry dictates how the system should be validated, sterilized, and IQ/OQ. Having to deal with a single nozzle is much simpler than designing a jet, especially in scale-up.

To determine whether the cell disruption occurs at the orifice entrance/exit or within the impingement zone, one of the nozzles was removed from the jet impinging device. The breakage of cell broth through a single nozzle was compared with the breakage produced by the opposing jets discharged from two nozzles.

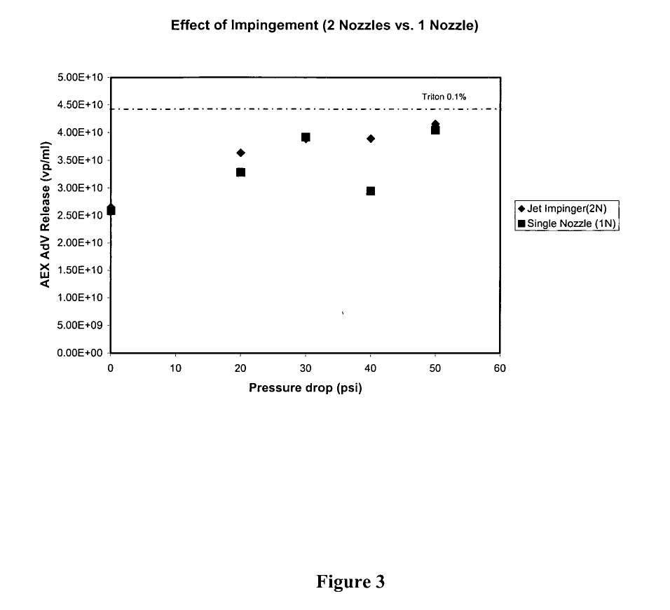

Cell cultures produced in 10 L bioreactors were provided for the experiments. A pressure tank equipped with a pressure regulator was used to control the flow rates flowing through the lysis device. The chosen pressures were 20, 30, 40, and 50 psi. The corresponding flows were channeled through 1 nozzle (a device according to the present invention) or 2 nozzles (jet impingement device), respectively. Lysates were clarified in a centrifuge at 4,000 rpm for 10 minutes, frozen at −70° C., and submitted subsequently for anion-exchange-chromatography (AEX) HPLC. The results were shown in FIG. 3.

The AEX data of the experiment in FIG. 3 indicate that the releases by the single nozzle device, especially that at 50 psi, are close to the maximum (the release by 0.1% Triton). Hence, the method according to the prevent invention is a very effective cell-disruption method.

Furthermore, the data suggest that the cell disruption happens at the orifice of the nozzle. At all the pressures, the release of adenovirus is similar in either case in the 1 nozzle and in the impingement set-up. The lower release of the single nozzle device obtained at 40 psi appears to be an outlier. If it is ignored, the trend of a greater release for increasing pressure is more realistic, which is confirmed by repeated experiment (data not shown).

### Example 2

### Effects of Orifice Size and Pressure on the Disruption of Mammalian Cells with a Single Nozzle Device

Experiments were conducted to understand the effect of nozzle diameters on the release of intracellular products such as adenovirus particles. Three experiments corresponding to three nozzle diameters were performed. Three orifice diameters were tested: 1, 2.1, and 3 mm.

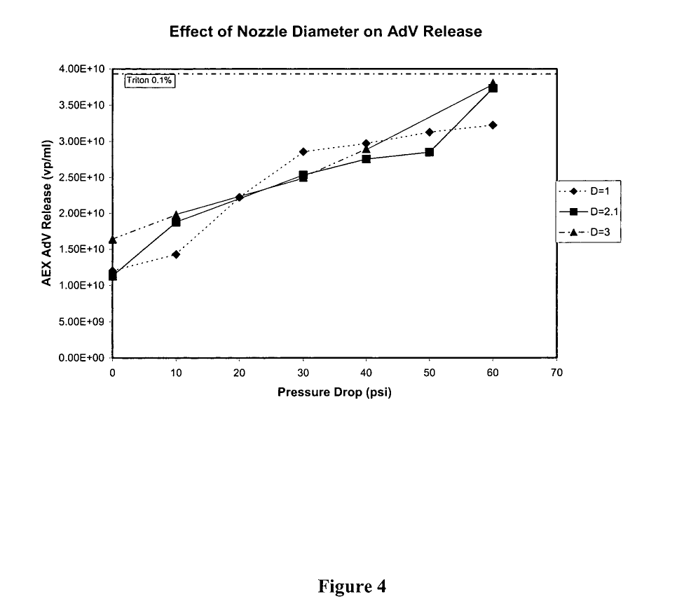

The flow rates were delivered by the following pressure drops: 10, 20, 30, 40, 50, and 60 psi. The device with a single nozzle was used to lyse mammalian cells (PER.C6 cells) suspended in a suspension. Lysates were collected, clarified, and analyzed using anion exchange chromatography (AEX). A sample was lysed with TritonX-100 to serve as a control. The AEX data are presented in FIG. 4.

This graph shows that the orifice diameter does not play an important role in the cell-disruption process, at the range from 1 mm to 3 mm. The three curves show a comparable release.

What is implicit in the results is the effect of linear velocity. At the same pressure drop, the flow rate through the nozzle with larger orifice is the highest while the linear velocity is the lowest. For example, at a pressure drop of 60 psi, the flow rate through the 1 mm nozzle is 1.2 L/min and the linear velocity is 25 m/sec. For the same pressure drop, the flow rate through the 3 mm I.D. nozzle is 7.0 L/min whereas the linear velocity is only 17 m/sec (data not shown).

By generating the same release at a lower linear velocity, i.e. at a lower shear rate, larger orifice diameters consist of other forms of shear. Extensional or elongational flows would compensate for the deficiency in shear stress. This experiment has been repeated to confirm these findings. Hence, the most important factor in causing the cell lysis is the pressure drop.

Other embodiments are within the following claims. While several embodiments have been shown and described, various modifications may be made without departing from the spirit and scope of the present invention.

What is claimed:

1. A method for disrupting cultured cells that lack a cell wall comprising passing the cells suspended in a suspension fluid through a nozzle at a low pressure, wherein the outflow of the nozzle does not impinge on the outflow of a second nozzle if multiple nozzles are present.

2. The method of claim 1 wherein the low pressure ranges from 1 to 100 psi.

3. The method of claim 1 wherein the low pressure ranges from 5 to 70 psi.

4. The method of claim 1 wherein the low pressure ranges from 10 to 60 psi.

5. The method of claim 1 wherein the nozzle has an orifice with a diameter ranging from 0.1 mm to 100 mm.

6. The method of claim 5 wherein the orifice has a diameter ranging from 0.5 mm to 10 mm.

7. The method of claim 1 wherein the orifice has a diameter ranging from 1 mm to 3 mm.

8. The method of claim 1 wherein the nozzle has a tapered or conical shape.

9. The method of claim 1 wherein the nozzle has both a tapered entrance and a tapered exit.

10. The method of claim 1 wherein the cells are animal cells.

11. The method of claim 9 wherein the animal cells are selected from the group consisting of: VERO cells, CHO cells, and diploid fibroblast cells.

12. A method according to claim 10 wherein the animal cells are MRC-5 diploid lung cells.

13. A method of harvesting a cell product contained within cells that does not have a cell wall comprising:
culturing the cells in a culture medium under culture conditions suited to bring about the production of the desired cell product;
suspending the cells in a suspension fluid;
passing the suspended cells through a nozzle at a low pressure, wherein the outflow of the nozzle does not impinge on the outflow of a second nozzle or any other impingement surface, so that the cells are disrupted at a pressure of from about 5 to 100 psi and the cell product is released; and
recovering the released cell product.

14. The method of claim 13 wherein the product is selected from the group consisting of a polysaccharide, a protein, and a virus.

15. A method of harvesting a virus grown in an animal cell comprising:
culturing animal cells infected with the virus;
suspending the animal cells containing the virus in a suspension fluid;
passing the suspended animal cells through a nozzle at a low pressure, wherein the outflow of the nozzle does not impinge on the outflow of a second nozzle if multiple nozzles are present, so that cells are disrupted and the virus is released; and
harvesting the released virus.

16. A method according to claim 12 wherein the virus is Varicella virus.
